(19) United States
(12) Patent Application Publication
(10) Pub. No.: US 2012/0150271 A1
(43) Pub. Date: Jun. 14, 2012

(54) BUDDY WIRE FOR THE CORONARY ARTERIES

(75) Inventors: DAVID R. FISCHELL, FAIR HAVEN, NJ (US); TIM A. FISCHELL, KALAMAZOO, MI (US); MARK POMERANZ, BERNARDSVILLE, NJ (US); ERIN M. FISCHELL, BELMONT, MA (US)

(73) Assignee: SVELTE MEDICAL SYSTEMS, INC., NEW PROVIDENCE, NJ (US)

(21) Appl. No.: 12/964,071

(22) Filed: Dec. 9, 2010

(51) Int. Cl. A61M 25/09; A61F 2/84

(52) U.S. Cl. 623/1.11; 604/528; 606/191

(57) ABSTRACT

A buddy wire guide wire has a wire section for guiding a catheter to a particular site in the human body. The buddy wire guide wire has a distal section with a distal end and an opposing proximal end. The wire section is attached to the proximal end of the distal section with the distal section being placed coaxially around the shaft of some existing medical device which has been inserted into the human body. The distal section permits the buddy wire to be advanced into the human body over the medical device by using the medical device as a guide.

### FIELD OF USE

This invention is in the field of percutaneous devices that are used to open a vessel of the human body.

### BACKGROUND OF THE INVENTION

In balloon angioplasty or stenting of vessels of the human body, it is often required to introduce a 2^{nd} guide wire (buddy wire) into the artery to assist in a number of clinical settings:
When, using fixed wire stent or balloon systems, there is a need to introduce a 2^{nd} balloon or stent into the artery. For example, if a dissection occurs after treatment of a stenosis with a fixed wire balloon angioplasty or stent delivery system, there is no separate guide wire that lies distal to the dissection to allow delivery of another stent or a balloon angioplasty catheter to the dissection site.
For cases in which the tortuosity of the vessel allows a flexible guide wire to pass through a stenosis in an artery, but it is not possible to advance a balloon angioplasty catheter or stent delivery system over the flexible guide wire. In this situation a buddy guide wire is often useful in “straightening” the severe curvature in the target vessel to assist in the successful delivery of the balloon angioplasty catheter or stent delivery catheter. With today's technology, this requires that one steer, and recross through the entire tortuous artery and pass the second (buddy) wire across the stenosis, alongside the first guidewire. This can be a difficult and time-consuming process.

### SUMMARY OF THE INVENTION

The present invention is a buddy guide wire having a rapid exchange distal portion designed to be placed over an existing guide wire or catheter and advanced into the human body using the existing wire or catheter as a guide. This will greatly simplify getting the buddy wire quickly to the site desired within a vessel of the human body. The embodiment of the present invention for use with an existing guide wire has a short structure attached to the distal end of a buddy wire. The structure may be a tube, a ring, a helical wire or any structure that can track over a guide wire or catheter and allow the buddy wire to follow the guide wire or catheter into the body. The structure is inserted over the proximal end of the existing guide wire (or catheter) and advanced distally using the existing guide wire as a guide. This can allow the rapid introduction of the buddy wire in a tortuous artery to promote the passage of a stent delivery system or balloon catheter to the site of a blockage. Ideally, the body of the buddy wire will be stiffer than the wire previously used to try to advance a balloon or stent to the desired site.

The embodiment of the present invention for use with an existing catheter system, e.g. a fixed wire stent delivery system such as that described by Fischell et al in U.S. Pat. Nos. 6,375,660, 6,936,065 and 7,011,673, would have the buddy wire have an attachment mechanism at its distal end designed to be attached to the proximal portion of the catheter outside of the body and then advanced over the catheter to the desired site within the body using the catheter as its “guide”. Examples of mechanisms to attach the buddy wire to the catheter shaft include
a tube having a slit that can be snapped onto the proximal shaft of the catheter
a tube with a slot in which the tube can be manually squeezed together to close the slot around the shaft of the catheter.
a helical wire that can be placed around the shaft of the catheter.

Thus it is an object of the present invention to have a guide wire having a mechanism that allows it to be advanced over a previously inserted guide wire into the human body to a desired site within a vessel of the human body.

Another object of the present invention to have a guide wire having a mechanism that allows it to be advanced over a previously inserted catheter into the human body to a desired site within a vessel of the human body

These and other objects and advantages of this invention will become obvious to a person of ordinary skill in this art upon reading of the detailed description of this invention including the associated drawings.

### BRIEF DESCRIPTION OF THE DRAWINGS

FIG. 1 is a 3 dimensional sketch of the distal end of the present invention buddy wire including a distal tube designed for advancement into the human body over a previously introduced guide wire to a desired site within a human body.

FIG. 2 is a 3 dimensional sketch of the distal end of the present invention buddy wire including a slotted distal tube designed to attach onto and be advanced over the shaft of a previously introduced catheter to a desired site within a human body.

FIG. 3 of the distal end of the present invention buddy wire including a slotted distal tube designed to be pressed into a closed (or nearly closed) tube over the shaft of a previously introduced catheter (or guide wire) and then advanced over the catheter to a desired site within the vessel of a human body.

FIG. 4 is a radial cross section at 4-4 of FIG. 3 representing the cross section of the distal end of the present invention buddy wire including a slotted distal tube designed to be pressed into a closed (or nearly closed) tube over the shaft of a previously introduced catheter (or guide wire) and then advanced over the catheter to a desired site within the vessel of a human body.

FIG. 5A is a radial cross section of the buddy wire system of FIGS. 3 and 4 showing it as it is begun to be pushed around the shaft of a catheter.

FIG. 5B is a radial cross section of the buddy wire system of FIGS. 3 and 4 showing it as it has been pushed over the shaft of a catheter but before it is squeezed closed over the shaft.

FIG. 5C is a radial cross section of the buddy wire system of FIGS. 3 and 4 showing it over the shaft of a catheter and after it has been squeezed closed (or nearly closed) to form a cylinder around the shaft of the catheter.

FIG. 6 is a 3 dimensional sketch of the distal end of the present invention buddy wire including a distal helical wire designed to attach onto and be advance over the shaft of a previously introduced catheter or guide wire to a desired site within the vessel of a human body.

FIG. 7 is a 3 dimensional sketch of the distal end of the present invention buddy wire including a distal helical wire shown after it has been attached onto the shaft of a previously introduced catheter or guide wire.

FIG. 8 is a 3 dimensional sketch of the distal end of the present invention buddy wire including a distal steerable guidewire extending in the distal direction beyond the helical wire section designed to attach onto and be advance over the shaft of a previously introduced catheter or guide wire to a desired site within the vessel of a human body.

### DETAILED DESCRIPTION OF THE DRAWINGS

The present invention is a buddy guide wire having a rapid exchange distal portion designed to be placed over an existing guide wire or catheter and advanced into the human body using the existing wire or catheter as a guide. This will greatly simplify getting the buddy wire quickly to the site desired within a vessel of the human body. An important use of the buddy wire is to allow the physician to quickly introduce a new catheter to a desired site following a procedural complication such as an intimal disection following balloon angioplasty of a coronary artery.

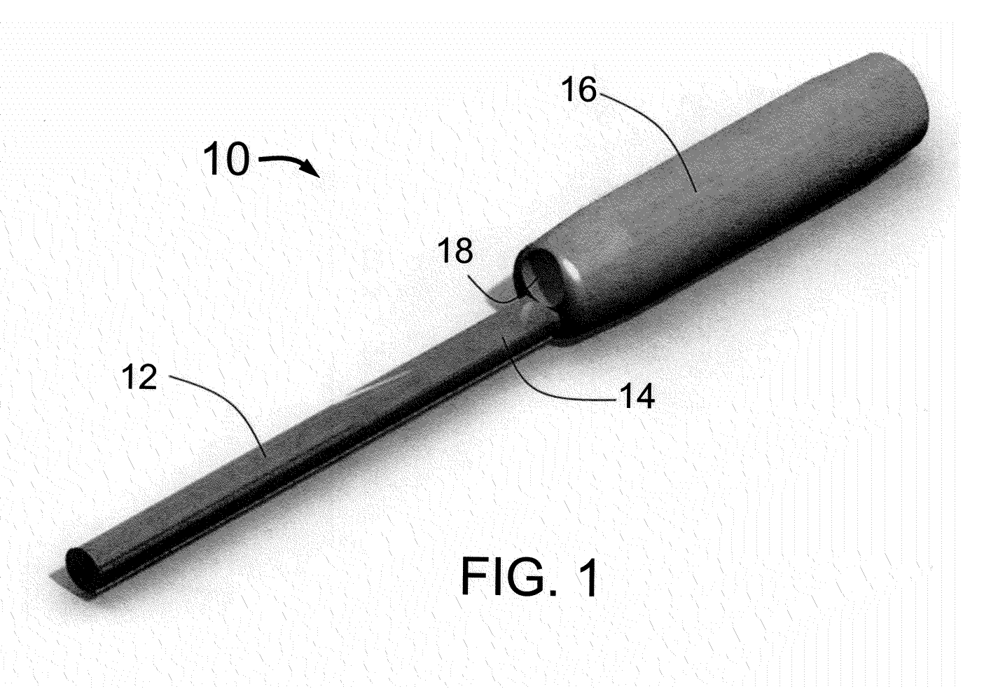

FIG. 1 is a 3 dimensional sketch of the distal end of the present invention buddy wire 10 including a distal tube 16 designed for advancement into the human body over a previously introduced guide wire to a desired site within a human body.

The buddy wire 10 includes a proximal wire 12 that extends for a length of up to 3 meters in a proximal direction, a tapered section 14 that is connected to the distal tube 16 having a lumen 18. The outside of the tube 16 should be formed from a soft plastic so that it will not cause injury as it is advanced into the body. The tube 16 would typically include a radiopaque marker or be formed from a radiopaque material such as tungsten filled urethane. The distal tube 16 would typically be adhesively attached or molded onto the distal end of the tapered section 14. The purpose of the tapered section 14 is to minimize the overall diameter of the distal tube 16 for a given diameter lumen 18 as a smaller diameter is easier to introduce through vessels of the human body. The lumen 18 would typically be slightly larger (for example 0.001″ larger) than the guidewire over which it is designed to be placed. Typical guide wires that might be used with the buddy wire 10 include guide wires of diameters 0.010″, 0.014″, 0.018″ and 0.035″.

The method that is used to place the buddy wire 10 around an existing guide wire is as follows:
1. Remove any catheter that is over the existing guide wire leaving only the existing wire advanced into the body.
2. Place the proximal end of the existing wire through the lumen 18 of the tube 16.
3. Advance the buddy wire 10 over the existing wire to the desired spot in the body.
4. Remove the existing wire
5. Advance a balloon angioplasty catheter or stent over the buddy wire.
6. Perform the procedure with the catheter.
7. Remove the system from the body.

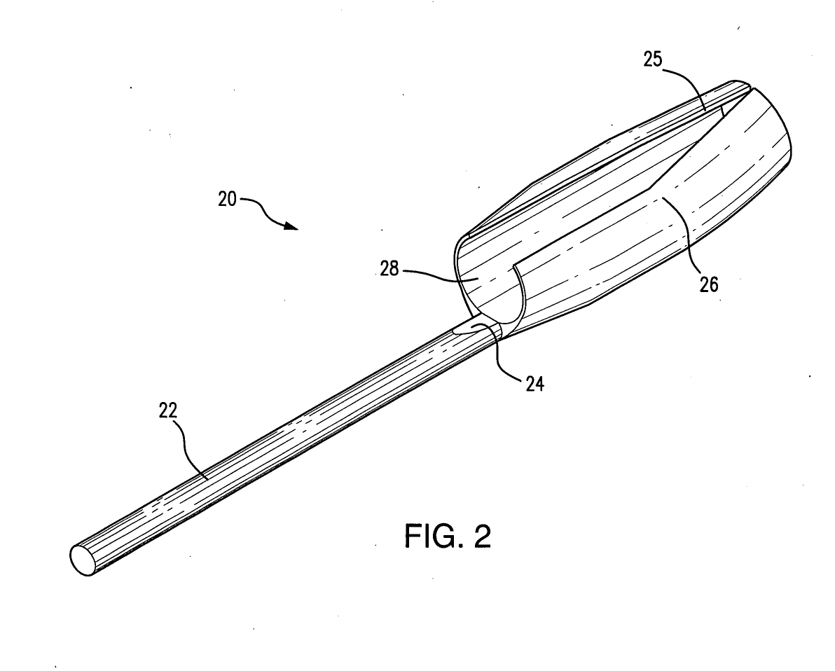

FIG. 2 is a 3 dimensional sketch of the distal end of another embodiment of the present invention buddy wire 20 including a slotted distal tube 26 designed to attach onto and be advanced over the shaft of a previously introduced catheter to a desired site within a human body. The buddy wire 20 includes a proximal wire 22 that extends for a length of up to 3 meters in a proximal direction, a tapered section 24 that is connected to the distal tube 26 having a lumen 28 and slot 25. The slot 25 is shown in FIG. 2 as being tapered with the smallest opening at its distal end. The tapering of the slot 25 is designed to provide better securement of the catheter within the lumen 28 as the buddy wire 20 is advanced through vessels of the human body and make it less likely that the catheter would come out of the slot 25. The outside of the tube 26 should be formed from a soft plastic so that it will not cause injury as it is advanced into the body. The tube 26 would typically include a radiopaque marker or be formed from a radiopaque material such as tungsten filled urethane.

The distal tube 26 would typically be adhesively attached or molded onto the distal end of the tapered section 24. The purpose of the tapered section 24 is to minimize the overall diameter of the distal tube 26 for a given diameter lumen 28 as a smaller diameter is easier to introduce through vessels of the human body.

The method that is used to place the buddy wire 20 around an existing catheter is as follows—
1. Slide the shaft of the proximal section of the existing catheter through the slot 25 into the lumen 28 of the distal tube 26.
2. Advance the buddy wire 20 over the existing catheter to the desired spot in the body.
3. Remove the existing catheter.
4. Advance a balloon angioplasty catheter or stent over the buddy wire.
5. Perform the procedure with the catheter.
6. Remove the system from the body.

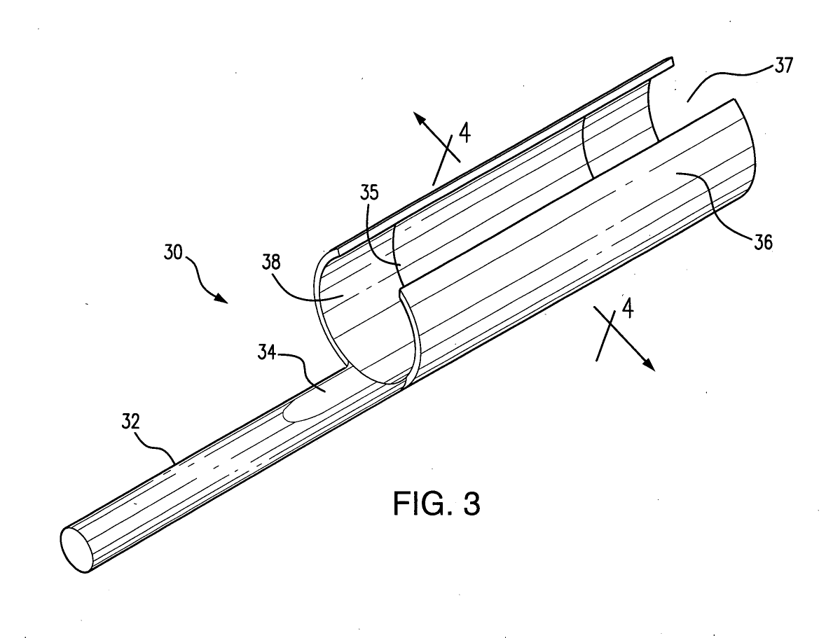

FIG. 3 of the distal end of the present invention buddy wire 30 including a slotted distal tube 36 designed to be pressed into a closed (or nearly closed) tube over the shaft of a previously introduced catheter (or guide wire) and then advanced over the catheter to a desired site within the vessel of a human body. The buddy wire 30 includes a proximal wire 32 that extends for a length of up to 3 meters in a proximal direction, a tapered section 34 that is connected to the distal tube 36 having a lumen 38 and slot 37. The outside of the tube 36 should be formed from a soft plastic so that it will not cause injury as it is advanced into the body. Centered in the inside of the tube 36 is a cut metal cylinder 35 that is attached to the plastic tube 36. The metal cylinder 35 would be typically welded to the tapered section of the wire 34. The tube 36 would typically include a radiopaque marker or be formed from a radiopaque material such as tungsten filled urethane. The method that is used to place the buddy wire 30 around an existing catheter (or guide wire) is as follows:
1. Slide the shaft of the proximal section of the existing catheter (or guide wire) through the slot 37 into the lumen 38 of the distal tube 36.
2. Squeeze the distal tube 36 closing the distal tube 36 over the catheter or guide wire.
3. Advance the buddy wire 30 over the existing catheter to the desired spot in the body.
4. Remove the existing catheter or guide wire.
5. Advance a balloon angioplasty catheter or stent over the buddy wire.
6. Perform the procedure with the catheter.
7. Remove the system from the body.

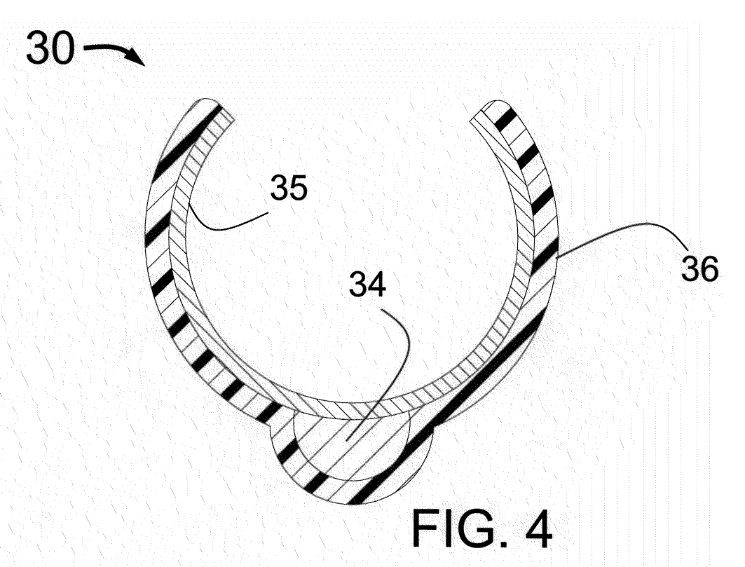

FIG. 4 is a radial cross section at 4-4 of FIG. 3 representing the cross section of the distal end of the present invention buddy wire 30 including a slotted distal tube 36 designed to be pressed into a closed (or nearly closed) tube over the shaft of a previously introduced catheter (or guide wire) and then advanced over the catheter to a desired site within the vessel of a human body. The plastic tube 36 is adhesively attached or molded over the metal cylinder 35 that is welded to the tapered section of the guide wire 34.

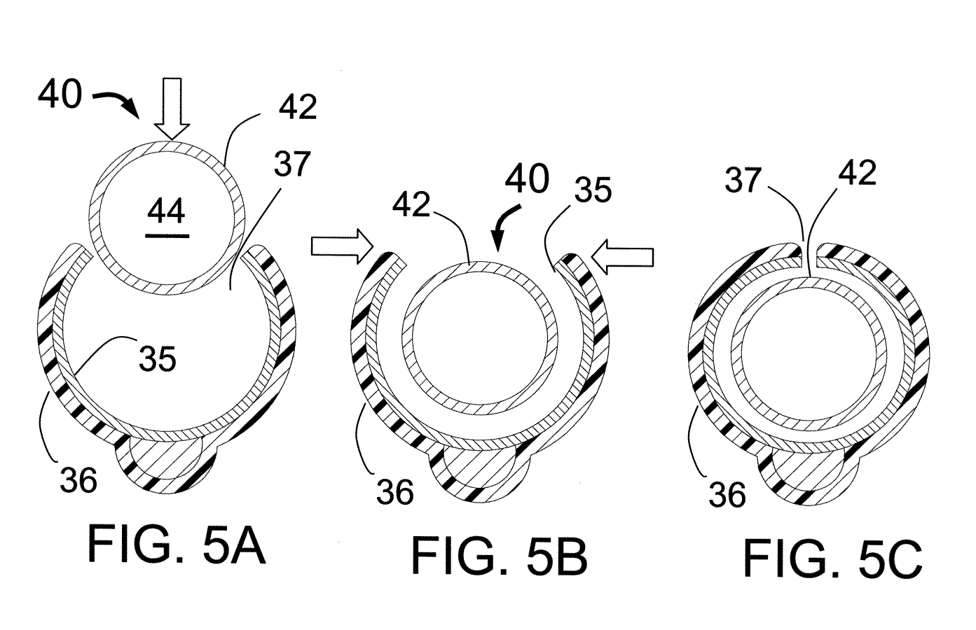

FIG. 5A is a radial cross section of the buddy wire 30 of FIGS. 3 and 4 with plastic tube 36 and metal cylinder 35 having slot 37 showing it as it is begun to be pushed around the shaft 42 with lumen 44 of the catheter 40. The arrow shows the direction in which the shaft 42 is pushed into the slot

FIG. 5B is a radial cross section of the buddy wire 30 of FIGS. 3 and 4 showing it as it has been pushed over the shaft 42 of the catheter 40 but before the tube 36 is squeezed closed over the shaft 42. The arrows show the direction in which the sides of the plastic tube 36 are squeezed together to form the configuration of FIG. 5C.

FIG. 5C is a radial cross section of the buddy wire 30 of FIGS. 3 and 4 showing it after it has been over the shaft of a catheter and after it has been squeezed closed (or nearly closed) to form a cylinder around the shaft of the catheter.

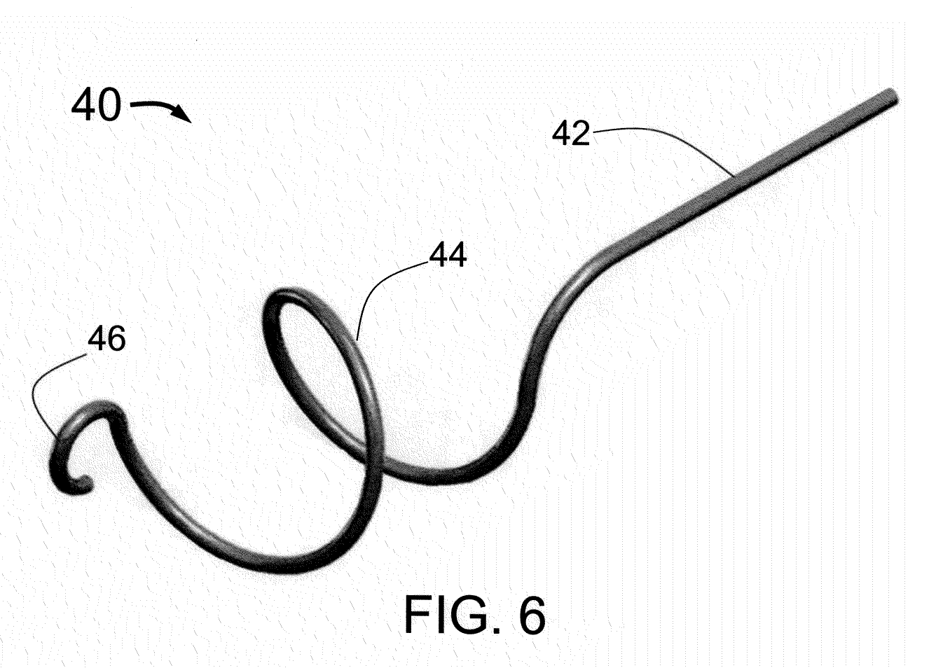

FIG. 6 is a 3 dimensional sketch of the distal end of another embodiment of the present invention buddy wire 40 including a proximal wire 42 attached to a distal helical wire 44 with distal end 46. The helical wire 44 is designed to attach around and be advanced over the shaft of a previously introduced catheter or guide wire to a desired site within the vessel of a human body. The buddy wire 40 has the advantage that the helical section 44 and distal end 46 are formed from a wire that is soft enough to allow it to straighten and be pulled out of the body through the guide wire lumen of any over the wire catheter system including over the wire stents and over the wire balloon angioplasty catheters. Thus this embodiment 40 after being introduced to the desired site within the body and for example, an over-the-wire stent is guided by the buddy wire 40 to or near the desired site within the body, a guide wire exchange can occur by pulling the buddy wire 40 out of the body in the proximal direction through the guide wire lumen of the over-the-wire catheter. After the buddy wire 40 is completely removed, a standard guide wire can be advanced through the guide wire lumen of the over-the-wire catheter and out of the distal end of the guide wire lumen completing the guide wire exchange. In this way the physician can now have in place a standard guide wire to use in continuing the procedure.

It is envisioned that the helical section 44 would be more bendable than the proximal wire 42 so it is more easily pulled out through a guide wire lumen in an over the wire catheter as well as to minimize the overall diameter of the helical section 44. To make the helical section 44 more bendable it can be formed from a softer material or heat treated to make it softer or it can be of smaller diameter than the proximal section 42. For example the helical section 44 it should be at least 0.001″ smaller in diameter than the proximal section 42.

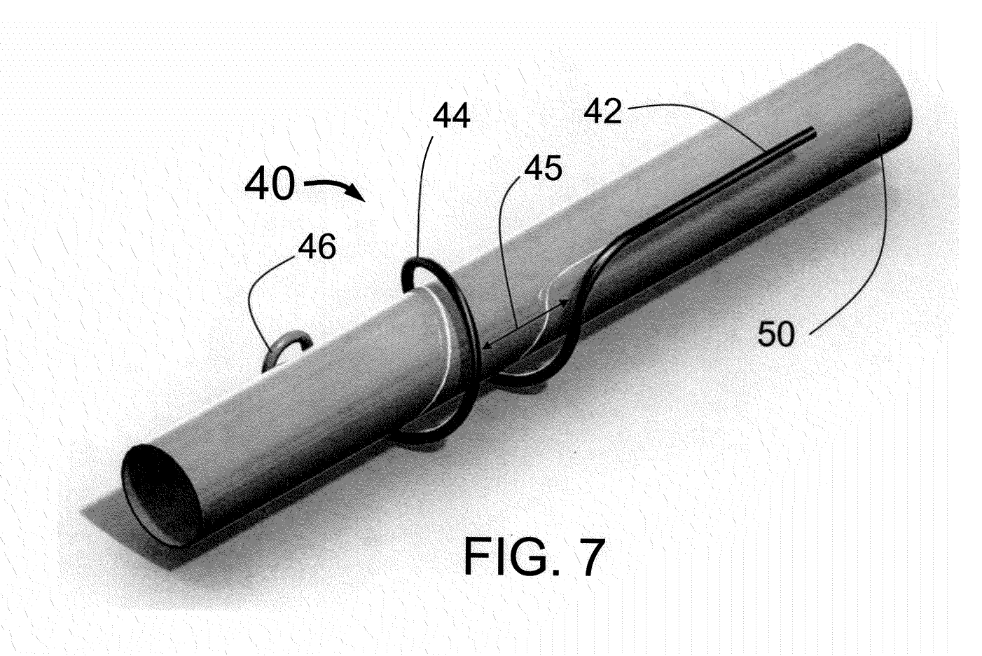

FIG. 7 is a 3 dimensional sketch of the distal end of the present invention buddy wire 40 including the distal helical wire 44 with distal end 46. The buddy wire 40 is shown after it has been attached over the shaft of a previously introduced catheter 50. The method for attaching the buddy wire 40 over the catheter 50 is to push the opening 45 in the helical section 44 of the buddy wire 40 against the catheter 50 at an angle just less than 90 degrees and rotating the helical section 44 winding it over the shaft of the catheter 50 until the helical section 44 goes completely around the catheter 50. The buddy wire 40 can then be advanced in the distal direction guided by the catheter 50.

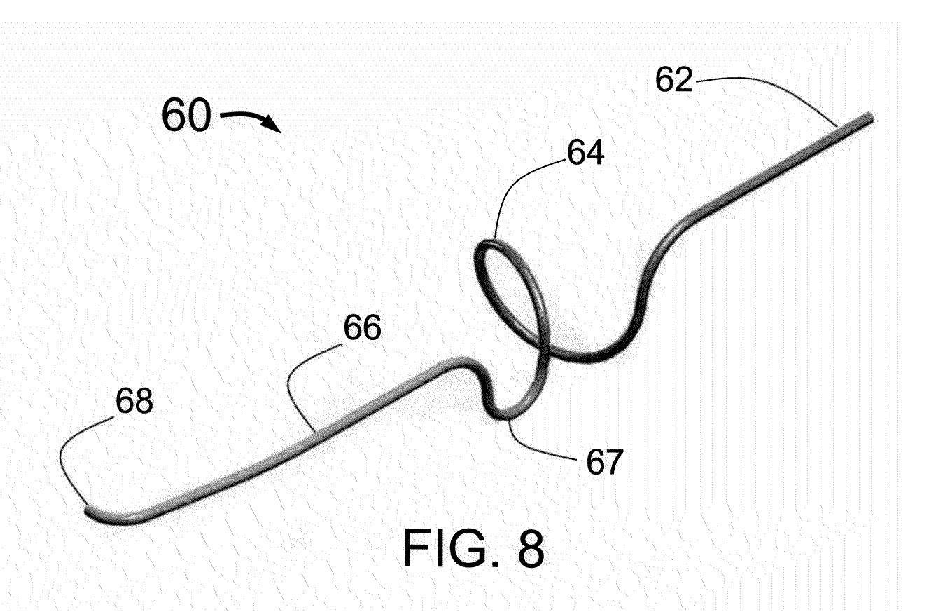

FIG. 8 is a 3 dimensional sketch of another embodiment of the distal end of the present invention buddy wire 60 including a distal flexible wire section 66 extending in the distal direction beyond a helical wire section 64. The distal flexible wire section 66 includes a formable distal end 68. The distal flexible wire section 66 should have the one or more of the properties of the distal section of a standard guide wire. These include the ability to shape or form the distal end 68 as well as the ability to steer the shaped end through vessels of the human body. Between the helical section 64 and the distal flexible wire section 66 is a transition section 67 that longitudinally aligns the distal section 66 with the proximal section 62 to enhance the ability to steer buddy wire 60 along the desired path in the vessels of the human body such as the coronary arteries. The helical wire section 64 is designed to attach onto and be advanced over the shaft of a previously introduced catheter or guide wire to a desired site within the vessel of a human body. The helical wire section 64 is attached at its proximal end to the distal end of the proximal wire 62. The distal flexible wire section 66 with formable distal end 68 may be constructed as current guide wires including a core wire and an outer layer that is either a wrapped wire or a polymer. The formable distal end 68 can be bent into the desire shape by the physician before the buddy wire 60 is inserted into the human body. This and all embodiments may also be lubricity coated.

Various other modifications, adaptations, and alternative designs are of course possible in light of the above teachings. Therefore, it should be understood at this time that within the scope of the appended claims the invention may be practiced otherwise than as specifically described herein.

What is claimed is:

1. A buddy wire guide wire including a wire section designed to guide a catheter to a desired site in the human body and a distal section, the distal section having a distal end and a proximal end, the wire section being attached to the proximal end of the distal section, the distal section designed further to be placed coaxially around the shaft of an existing medical device that has been inserted into a human body, the distal section allowing the buddy wire to be advanced into the human body over the medical device, using the medical device as a guide.

2. The buddy wire of claim 1 where the existing medical device is an existing guide wire that has been advanced to a site within the human body.

3. The buddy wire of claim 2 where the distal section includes a tubular structure that is coaxially surrounds the existing guide wire, the tubular structure being is movable in the longitudinal direction over the existing guide wire.

4. The buddy wire of claim 1 where the medical device is a catheter.

5. The buddy wire of claim 4 where the catheter is a balloon angioplasty catheter.

6. The buddy wire of claim 4 where the catheter is a stent delivery catheter including a deployable stent.

7. The buddy wire of claim 4 where the distal section includes a slot that will attach onto the shaft of the catheter allowing the buddy wire to be advanced into the body.

8. The buddy wire of claim 4 where the distal section includes a cylinder having a longitudinal opening into which the shaft of the catheter can be inserted, the cylinder being plastically deformable to close the longitudinal opening so that the cylinder coaxially surrounds the shaft of the catheter, the buddy wire then being advanced over the catheter into the human body.

9. The buddy wire of claim 1 where the distal section includes a helical wire, the helical wire being designed to be attached onto the shaft of the existing medical device and advanced over the medical device to the desired site in the human body.

10. The buddy wire of claim 9 where the helical wire has a diameter at least 0.001″ less than the wire section proximal to the helical wire.

11. The buddy wire of claim 9 further including a distal flexible wire section, the proximal end of the distal flexible wire section being attached to the distal end of the helical wire.

12. The buddy wire of claim 11 where the distal flexible wire section has a formable distal end.

13. The buddy wire of claim 9, the helical wire further being deformable so that after removal of the existing medical device and insertion over the wire section of the buddy wire of a second medical device having a guide wire lumen that runs the length of the second medical device, the buddy wire then being removable from the human body through the guide wire lumen of the second medical device.

14. The buddy wire of claim 13 where the medical device is an over-the-wire stent delivery catheter including an expandable stent.

15. A method for inserting a guide wire into a vessel of the human body having a previously inserted medical device, the medical device having a proximal end that lies outside the body and a distal end that lies within a vessel of the human body, the method being for the purpose of guiding a catheter into the vessel, the method comprising the steps of:
a. placing a buddy wire guide wire having a distal section and a proximal wire section such that the distal section lies coaxially over the portion of the medical device that lies outside the body;
b. advancing the buddy wire over the medical device in the distal direction until the distal section of the buddy wire is at the desired location in the vessel of the human body;
c. placing the proximal end of the wire section of the buddy wire into the guide wire lumen of a catheter having a distal end;
d. advancing the catheter over the buddy wire until the distal end of the catheter is at the desired location in the vessel.

16. The method of claim 15 where the medical device is chosen from the group of a guide wire or a catheter

17. The method of claim 16 where the catheter is chosen from the group of a balloon angioplasty catheter or a stent delivery catheter.

18. The method of claim 15 where the catheter guide wire lumen extends outside of the body when the distal end of the catheter is at the desired location in the vessel, the method including step 5 of removing the buddy wire from the body through the guide wire lumen.

19. The method of claim 15 further including a step between steps b and c where the medical device is removed from the human body before the catheter is placed over the buddy wire and advanced into the human body.
